(19) United States
(12) Patent Application Publication
(10) Pub. No.: US 2006/0111404 A1
(43) Pub. Date: May 25, 2006

(54) Salts of N-[2-({(3R)-1-[trans-4-hydroxy-4-(6-methoxypyridin-3-yl)-cyclohexyl]pyrrolidin-3-yl}amino)-2-oxoethyl]-3-(trifluoromethyl)benzamide

(75) Inventors: Chu-Biao Xue, Hockessin, DE (US); Brian W. Metcalf, Moraga, CA (US); Hui-Yin Harry Li, Hockessin, DE (US); Hao Feng, Aston, PA (US); Joseph Glenn, Sicklerville, NJ (US)

Correspondence Address: COZEN O' CONNOR, P.C., 1900 MARKET STREET, PHILADELPHIA, PA, 19103-3508, US

(73) Assignee: INCYTE CORPORATION

(21) Appl. No.: 11/284,220

(22) Filed: Nov. 21, 2005

Related U.S. Application Data
(60) Provisional application No. 60/699,637, filed on Jul. 15, 2005.
(60) Provisional application No. 60/630,146, filed on Nov. 22, 2004.

(51) Int. Cl. A61K 31/4439; C07D 403/02

(52) U.S. Cl. 514/343; 546/276.4

(57) ABSTRACT

The present invention pertains to bis(methanesulfonic acid), bis(ethanesulfonic acid), and camphoric acid salts of chemokine receptor inhibitor N-[2-({(3R)-1-[trans-4-hydroxy-4-(6-methoxypyridin-3-yl)-cyclohexyl]-pyrrolidin-3-yl}amino)-2-oxoethyl]-3-(trifluoromethyl)-benzamide, methods of preparing the same, and methods of using the same.

### CROSS-REFERENCE TO RELATED APPLICATIONS

This application claims the benefit of U.S. Ser. Nos. 60/630,146, filed Nov. 22, 2004 and 60/699,637, filed Jul. 15, 2005, the disclosures of each of which are incorporated herein by reference in their entireties.

### FIELD OF THE INVENTION

The present invention pertains to pharmaceutically acceptable salts of chemokine receptor inhibitor N-[2-({(3R)-1-[trans-4-hydroxy-4-(6-methoxypyridin-3-yl)-cyclohexyl]-pyrrolidin-3-yl}amino)-2-oxoethyl]-3-(trifluoromethyl)-benzamide, methods of preparing the same, and methods of using the same.

### BACKGROUND OF THE INVENTION

N-[2-({(3R)-1-[trans-4-hydroxy-4-(6-methoxypyridin-3-yl)-cyclohexyl]pyrrolidin-3-yl}amino)-2-oxoethyl]-3-(trifluoromethyl)-benzamide having Formula I: 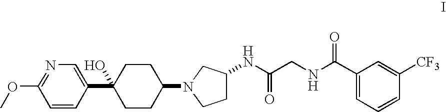
is a potent chemokine receptor antagonist, particularly with respect to CCR-type chemokine receptors such as CCR2. The compound of Formula I as well as its preparation and use have been described in WO 04/50024, which is incorporated herein by reference in its entirety. For the manufacture, purification, and formulation of a drug, it is typically advantageous to employ a form of the drug having superior stability or other desirable formulation property exhibited by, for example, one or more salt or crystalline forms of the drug. Accordingly, the salt forms of the compound of Formula I provided herein help satisfy the ongoing need for new stable forms of chemokine receptor inhibitors.

### SUMMARY OF THE INVENTION

The present invention provides, inter alia, a pharmaceutically acceptable salt of a compound of Formula I: 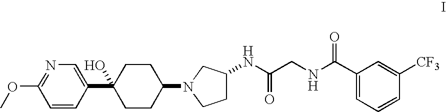
wherein the salt is a bis(methanesulfonic acid) salt, bis(ethanesulfonic acid) salt, or camphoric acid salt.

The present invention further provides methods for the preparation of salts of the invention.

The present invention further provides compositions comprising a salt of the invention and a pharmaceutically acceptable carrier.

The present invention further provides methods of modulating activity of a chemokine receptor comprising contacting the chemokine receptor with a salt of the invention.

The present invention further provides methods of treating a disease associated with expression or activity of a chemokine receptor in a patient comprising administering to the patient a therapeutically effective amount of a salt of the invention.

### BRIEF DESCRIPTION OF THE DRAWINGS

FIG. 1 depicts a differential scanning calorimetry (DSC) thermogram for a crystalline bis(methanesulfonic acid) salt form of the compound of Formula I (50-250° C. @ 10°/min).

FIG. 2 depicts an X-ray powder diffraction pattern for a crystalline bis(methanesulfonic acid) salt form of the compound of Formula I (Cu/1.54060 Å; 40.0 kV; 40.0 mA; 3-45° 2θ scan range; 0.020 step; 1.00° slit).

FIG. 3 depicts a differential scanning calorimetry (DSC) thermogram for a crystalline bis(ethanesulfonic acid) salt form of the compound of Formula I (50-250° C. @ 10°/min).

FIG. 4 depicts an X-ray powder diffraction pattern for a crystalline bis(ethanesulfonic acid) salt form of the compound of Formula I (Cu; 45.0 kV; 40.0 mA; 2-59° 2θ scan range; 0.030 step).

FIG. 5 depicts a differential scanning calorimetry (DSC) thermogram for a crystalline camphoric acid salt form of the compound of Formula I (50-250° C. @ 10°/min).

FIG. 6 depicts an X-ray powder diffraction pattern for a crystalline camphoric acid salt form of the compound of Formula I (Cu; 45 kV; 40 mA; 2-59° 2θ scan range; 0.030 step).

### DETAILED DESCRIPTION

The present invention provides, inter alia, a pharmaceutically acceptable salt of a compound of Formula I: 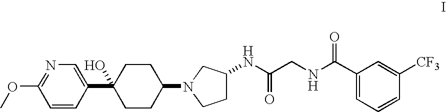
wherein the salt is a bis(methanesulfonic acid) salt, bis(ethanesulfonic acid) salt, or camphoric acid salt. The salts of the invention can be in amorphous or crystalline form, or a mixture thereof. In some embodiments, the salts of the invention are crystalline, including crystalline forms which are anhydrous, hydrated, non-solvated, or solvated. Example hydrates include hemihydrates, monohydrates, dihydrates, and the like. In some embodiments, the salt forms are anhydrous and non-solvated. The salts of the invention are particularly advantageous for use in pharmaceutical formulations because the salts can be isolated in crystalline form, thereby facilitating preparation, purification, and formulation of the drug.

The salts of the invention can be prepared by any suitable method for the preparation of acid addition salts. For example, the free base compound of Formula I can be combined with the desired acid in a solvent or in a melt. Alternatively, an acid addition salt of Formula I can be converted to a different acid addition salt by anion exchange. Salts of the invention which are prepared in a solvent system can be isolated by precipitation from the solvent. Precipitation and/or crystallization can be induced, for example, by evaporation, reduction of temperature, addition of anti-solvent, or combinations thereof.

The salts of the invention can be provided in a composition. In some embodiments, the composition contains a salt of the invention in an amount greater than about 1%, greater than about 5%, greater than about 10%, greater than about 25%, greater than about 50%, greater than about 60%, greater than about 70%, greater than about 80%, greater than about 90%, greater than about 95% by weight %, greater than about 98% by weight %, or greater than about 99% by weight. In some embodiments, the composition consists essentially of a salt of the invention. In some embodiments, the composition comprises a salt of the invention and a pharmaceutically acceptable carrier.

Bis(Methanesulfonic Acid) Salt

The bis(methanesulfonic acid) salt of the compound of Formula I can be prepared by any suitable method for preparation of methanesulfonic acid addition salts. For example, the compound of Formula I can be combined with methanesulfonic acid (e.g., about 2 eq or more) in a crystallizing solvent and the resulting salt can be isolated by precipitating the salt from solution, such as by addition of an anti-solvent.

The crystallizing solvent can contain any solvent or mixture of solvents capable of at least partially dissolving the compound of Formula I. In some embodiments, the crystallizing solvent contains a mixture of water, alcohol and ketone. Suitable alcohols include methanol, ethanol, 2-nitroethanol, 2-fluoroethanol, 2,2,2-trifluoroethanol, ethylene glycol, 1-propanol, isopropanol (isopropyl alcohol, 2-propanol), 2-methoxyethanol, 1-butanol, 2-butanol, i-butyl alcohol, t-butyl alcohol, 2-ethoxyethanol, diethylene glycol, 1-, 2-, or 3- pentanol, neo-pentyl alcohol, t-pentyl alcohol, diethylene glycol monomethyl ether, diethylene glycol monoethyl ether, cyclohexanol, benzyl alcohol, phenol, or glycerol. In some embodiments, the alcohol contains methanol, ethanol, l-propanol, or isopropanol. In some embodiments, the alcohol contains isopropanol. Suitable ketones include acetone, methyl ethyl ketone, diethylketone, methyl isobutyl ketone, and the like. In some embodiments, the ketone is methyl isobutyl ketone.

In some embodiments, the crystallizing solvent contains water and alcohol in a volume ratio of about 1:2 to about 1:20, about 1:5 to about 1:12, or about 1:9.

The precipitation and/or crystallization of the bis(methanesulfonic acid) salt, in some embodiments, is induced by the addition of anti-solvent. A suitable anti-solvent can contain any solvent in which the salt is poorly soluble such as a ketone (e.g., methyl isobutyl ketone).

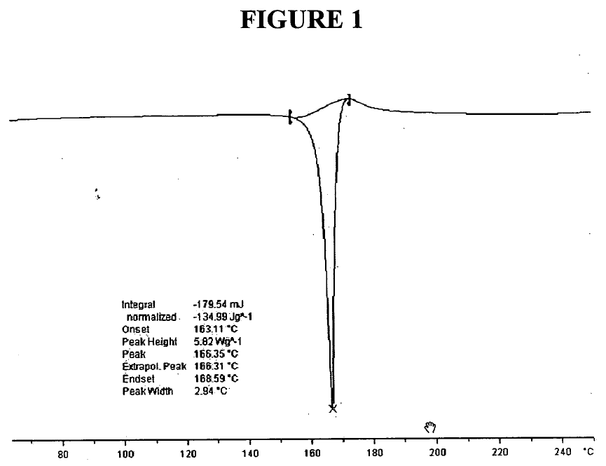

Crystalline bis(methanesulfonic acid) salt forms of the compound of Formula I can be identified by their unique signatures with respect to, for example, differential scanning calorimetry (DSC), X-ray powder diffraction, and other solid state methods such as FT-IR and solid state NMR. In some embodiments, the crystalline bis(methane sulfonic acid) salt can be characterized by the DSC trace substantially as shown in FIG. 1 having, as a prominent feature, an endotherm at about 166° C. The term “substantially” in this instance indicates that features such as endotherms, exotherms, baseline shifts, etc. can vary about±4° C. For DSC, it is known that the temperatures observed will depend upon the rate of temperature change as well as sample preparation technique and the particular instrument employed. Thus, the values reported herein relating to DSC thermograms can vary by plus or minus about 4° C.

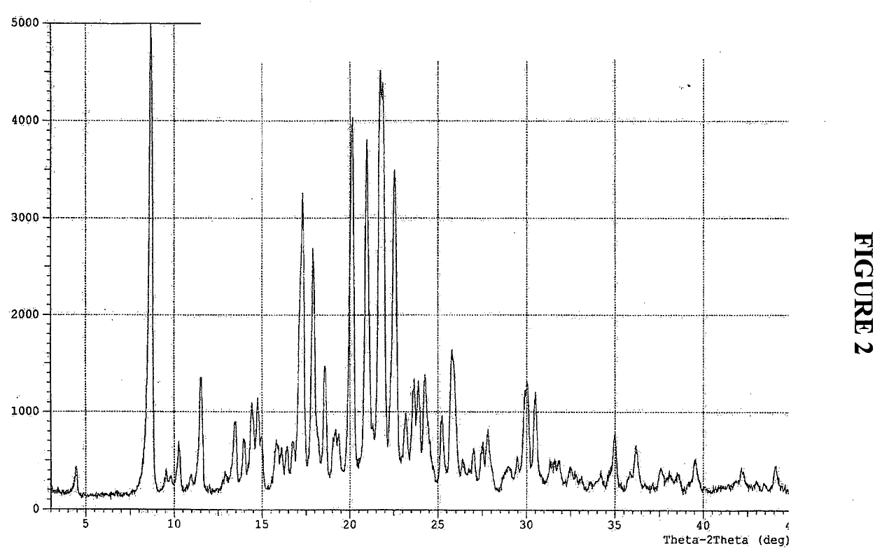

The crystalline bis(methanesulfonic acid) salt can also be identified by the X-ray powder diffraction (XRPD) pattern substantially as shown in FIG. 2. Major peaks from the XRPD pattern are listed below in Table 1. The term “substantially” in this instance indicates that 2-theta values for individual peaks can vary by about±0.2°. The relative intensities of the peaks can vary, depending upon the sample preparation technique, the sample mounting procedure and the particular instrument employed. Moreover, instrument variation and other factors can affect the 2-theta values. Therefore, the peak assignments can vary, as indicated above, by plus or minus about 0.2°.

In some embodiments, the crystalline bis(methanesulfonic acid) salt form of the compound of Formula I has an XRPD pattern having at least 3 peaks, in terms of 2θ, selected from Table 1. In some embodiments, the bis(methanesulfonic acid) salt has an XRPD pattern having peaks, in terms of 2θ, at about 8.7° and about 21.8°. In some embodiments, the bis(methanesulfonic acid) salt has an XRPD pattern having peaks, in terms of 2θ, at about 8.7°, about 21.8°, about 20.1° and about 20.9°. In some embodiments, the bis(methanesulfonic acid) salt has an XRPD pattern having peaks, in terms of 2θ, at about 8.7°, about 21.8°, about 20.1°, about 20.9°, about 22.5°, and about 17.2°.

TABLE 1

| Observed Peak, | Intensity |
|---|---|
| 2θ (°) | (CPS) |
| 8.7 | 1881 |
| 11.5 | 464 |
| 14.4 | 340 |
| 14.7 | 343 |
| 17.2 | 1106 |
| 17.9 | 893 |
| 18.6 | 461 |
| 20.1 | 1431 |
| 20.9 | 1301 |
| 21.8 | 1579 |
| 22.5 | 1199 |
| 23.6 | 340 |
| 23.9 | 342 |
| 24.3 | 405 |
| 25.8 | 521 |
| 29.9 | 410 |
| 30.5 | 379 |

Bis(Ethanesuifonic Acid) Salt

The bis(ethanesulfonic acid) salt of the compound of Formula I can be prepared by any suitable method for preparation of ethanesulfonic acid addition salts. For example, the compound of Formula I can be combined with ethanesulfonic acid (e.g., about 2 eq or more) in a crystallizing solvent and the resulting salt can be isolated by precipitating the salt from solution.

The crystallizing solvent can contain any solvent or mixture of solvents capable of at least partially dissolving the compound of Formula I. In some embodiments, the crystallizing solvent contains an alcohol. Suitable alcohols include methanol, ethanol, 2-nitroethanol, 2-fluoroethanol, 2,2,2-trifluoroethanol, ethylene glycol, 1-propanol, isopropanol (isopropyl alcohol, 2-propanol), 2-methoxyethanol, 1-butanol, 2-butanol, i-butyl alcohol, t-butyl alcohol, 2-ethoxyethanol, diethylene glycol, 1-, 2-, or 3- pentanol, neo-pentyl alcohol, t-pentyl alcohol, diethylene glycol monomethyl ether, diethylene glycol monoethyl ether, cyclohexanol, benzyl alcohol, phenol, or glycerol. In some embodiments, the alcohol contains isopropanol.

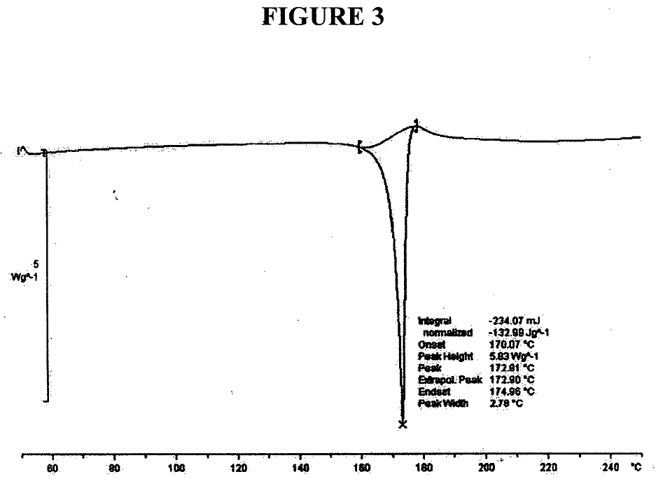

The crystalline bis(ethanesulfonic acid) salt of the compound of Formula I can be identified by its unique signatures with respect to, for example, differential scanning calorimetry (DSC), X-ray powder diffraction, and other solid state methods such as FT-IR and solid state NMR. In some embodiments, the crystalline bis(ethanesulfonic acid) salt can be characterized by the DSC trace substantially as shown in FIG. 3 having, as a prominent feature, an endotherm at about 173° C. The term “substantially” in this instance indicates that features such as endotherms, exotherms, baseline shifts, etc. can vary about±4° C. For DSC, it is known that the temperatures observed will depend upon the rate of temperature change as well as sample preparation technique and the particular instrument employed. Thus, the values reported herein relating to DSC thermograms can vary by plus or minus about 4° C.

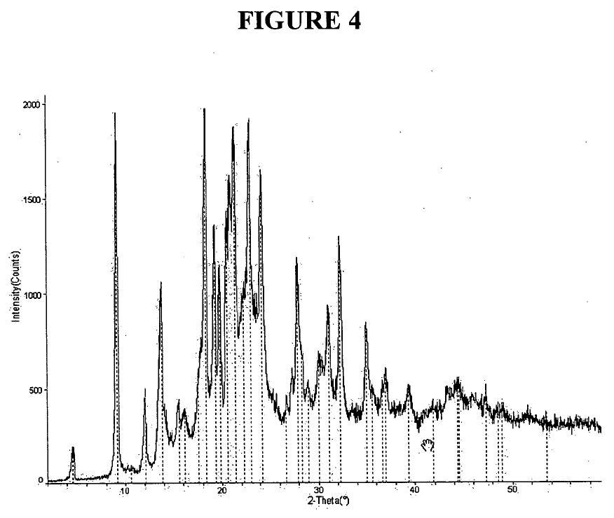

The crystalline bis(ethanesulfonic acid) salt can also be identified by the X-ray powder diffraction (XRPD) pattern substantially as shown in FIG. 4. Major peaks from the XRPD are listed below in Table 2. The term “substantially” in this instance indicates-that 2-theta values for individual peaks can vary by about±0.20. The relative intensities of the peaks can vary, depending upon the sample preparation technique, the sample mounting procedure and the particular instrument employed. Moreover, instrument variation and other factors can affect the 2-theta values. Therefore, the peak assignments can vary, as indicated above, by plus or minus about 0.2°.

In some embodiments, the crystalline bis(ethanesulfonic acid) salt form of the compound of Formula I has an XRPD pattern having at least 3 peaks, in terms of 2θ, selected from Table 2 (CPS less than 1000=“+;” CPS of 1000 to 1500=“++;” CPS greater than 1500=“+++”). In some embodiments, the bis(ethanesulfonic acid) salt has an XRPD pattern having at least one peak, in terms of 2θ, at about 9.2°. In some embodiments, the bis(ethanesulfonic acid) salt has an XRPD pattern having peaks, in terms of 2θ, at about 9.2°, about 12.1, and about 18.3. In some embodiments, the bis(ethanesulfonic acid) salt has an XRPD pattern having peaks, in terms of 2θ, at about 9.2°, about 12.10, about 13.8°, about 18.3°, about 19.3°, and about 19.8°.

TABLE 2

| Observed Peak, | Intensity |
|---|---|
| 2θ (°) | (CPS) |
| 9.2 | +++ |
| 12.1 | + |
| 13.8 | ++ |
| 18.3 | +++ |
| 19.3 | ++ |
| 19.8 | ++ |
| 20.6 | +++ |
| 21.4 | +++ |
| 23.0 | +++ |
| 24.2 | +++ |
| 27.9 | ++ |
| 32.3 | ++ |
| 35.0 | + |

Camphoric Acid Salt

The camphoric acid salt of the compound of Formula I can be prepared by any suitable method for preparation of camphoric acid addition salts. For example, the compound of Formula I can be combined with camphoric acid (e.g., about 1 eq or more) in a crystallizing solvent and the resulting salt can be isolated by precipitating the salt from solution.

The crystallizing solvent can contain any solvent or mixture of solvents capable of at least partially dissolving the compound of Formula I. In some embodiments, the crystallizing solvent contains an ester. Suitable esters include ethyl acetate, methyl acetate, and ethyl formate. In some embodiments, the crystallizing solvent contains ethyl acetate.

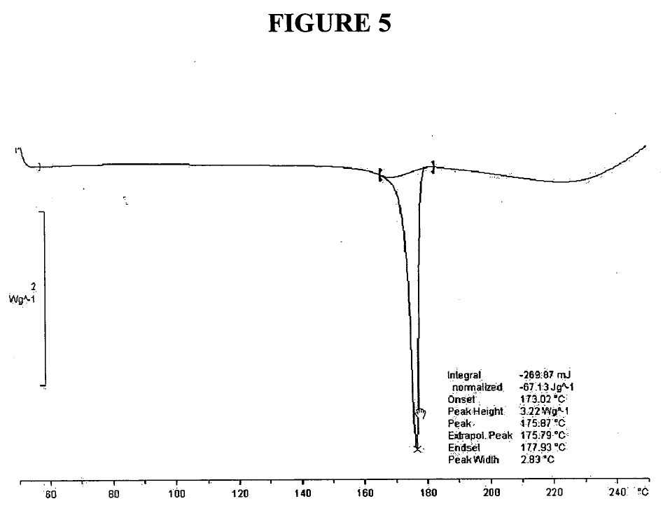

The crystalline camphoric acid salt of the compound of Formula I can be identified by its unique signatures with respect to, for example, differential scanning calorimetry (DSC), X-ray powder diffraction, and other solid state methods such as FT-IR and solid state NMR. In some embodiments, the crystalline camphoric acid salt can be characterized by the DSC trace substantially as shown in FIG. 5 having, as a prominent feature, an endotherm at about 176° C. The term “substantially” in this instance indicates that features such as endotherms, exotherms, baseline shifts, etc. can vary about±4° C. For DSC, it is known that the temperatures observed will depend upon the rate of temperature change as well as sample preparation technique and the particular instrument employed. Thus, the values reported herein relating to DSC thermograms can vary by plus or minus about 4° C.

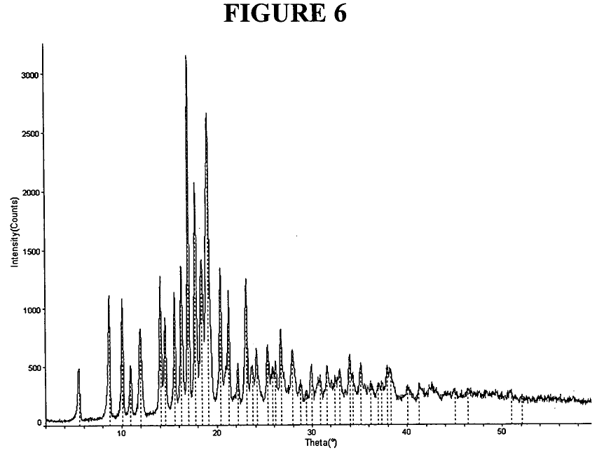

In some embodiments, the crystalline camphoric acid salt form is identified by the X-ray powder diffraction (XRPD) pattern substantially as shown in FIG. 6. Major peaks from the XRPD are listed below in Table 3. The term “substantially” in this instance indicates that 2-theta values for individual peaks can vary by about±0.2°. The relative intensities of the peaks can vary, depending upon the sample preparation technique, the sample mounting procedure and the particular instrument employed. Moreover, instrument variation and other factors can affect the 2-theta values. Therefore, the peak assignments can vary, as indicated above, by plus or minus about 0.2°.

In some embodiments, the camphoric acid salt of the compound of Formula I has an XRPD pattern having at least 3 peaks, in terms of 2θ, selected from Table 3 (CPS less than 1000=“+;” CPS of 1000 to 1500=“++;” CPS greater than 1500=“+++”). In some embodiments, the camphoric acid salt has an XRPD pattern having peaks, in terms of 2θ, at about 17.0° and about 19.1°. In some embodiments, the camphoric acid salt has an XRPD pattern having peaks, in terms of 2θ, at about 17.0°, about 19.1°, about 17.8° and about 14.1°. In some embodiments, the camphoric acid salt has an XRPD pattern having peaks, in terms of 2θ, at about 17.0°, about 19.1°, about 17.8°, about 14.1°, about 16.3°, and about 18.4°. In some embodiments, the camphoric acid salt has an XRPD pattern having peaks, in terms of 2θ, at about 17.0°, about 19.1°, about 17.8°, about 14.1°, about 16.3°, about 18.4°, about 10.1° and about 11.7°.

TABLE 3

| Observed Peak, | Intensity |
|---|---|
| 2θ (°) | (CPS) |
| 5.5 | + |
| 8.7 | ++ |
| 10.1 | ++ |
| 10.9 | + |
| 11.7 | + |
| 14.1 | ++ |
| 14.6 | + |
| 15.6 | ++ |
| 16.3 | ++ |
| 17.0 | +++ |
| 17.8 | +++ |
| 18.4 | ++ |
| 19.1 | +++ |
| 20.4 | ++ |
| 21.2 | ++ |
| 23.1 | ++ |
| 25.3 | + |
| 26.7 | + |
| 28.0 | + |
| 30.0 | + |

Methods

In some embodiments, salts of the invention can modulate activity of one or more chemokine receptors. The term “modulate” is meant to refer to an ability to increase or decrease activity of a receptor. Accordingly, salts of the invention can be used in methods of modulating a chemokine receptor by contacting the receptor with any one or more of the compounds or compositions described herein. In some embodiments, salts of the present invention can act as inhibitors of chemokine receptors. In further embodiments, the salts of the invention can be used to modulate activity of a chemokine receptor in an individual in need of modulation of the receptor by administering a modulating amount of a salt of the invention.

Chemokine receptors to which the present salts bind and/or modulate include any chemokine receptor. In some embodiments, the chemokine receptor belongs to the CC family of chemokine receptors including, for example, CCR1, CCR2, CCR3, CCR4, CCR5, CCR6, CCR7, CCR8, and CCR10. In some embodiments, the chemokine receptor is CCR2.

The present invention further provides methods of treating a chemokine receptor-associated disease or disorder in an individual (e.g., patient) by administering to the individual in need of such treatment a therapeutically effective amount or dose of a salt of the present invention or a pharmaceutical composition thereof. A chemokine receptor-associated disease can include any disease, disorder or condition that is directly or indirectly linked to expression, overexpression, activity or abnormal activity of the chemokine receptor. A chemokine receptor-associated disease can also include any disease, disorder or condition that can be prevented, ameliorated, or cured by modulating chemokine receptor activity.

Example chemokine receptor-associated diseases, disorders and conditions include inflammation and inflammatory diseases, metabolic diseases, immune disorders and cancer. In some embodiments, the chemokine receptor-associated disease is a viral infection such as HIV infection or AIDS. Example inflammatory diseases include diseases having an inflammatory component such as asthma, seasonal and perennial allergic rhinitis, sinusitis, conjunctivitis, age-related macular degeneration, food allergy, scombroid poisoning, psoriasis, urticaria, pruritus, eczema, inflammatory bowel disease, thrombotic disease, otitis media, liver cirrhosis, cardiac disease, Alzheimer's disease, sepsis, restenosis, atherosclerosis, multiple sclerosis, Crohn's disease, ulcerative colitis, hypersensitivity lung diseases, drug-induced pulmonary fibrosis, chronic obstructive pulmonary disease (COPD), rheumatoid arthritis, and nephritis, ulcerative colitis, atopic dermatitis, stroke, acute nerve injury, sarcoidosis, hepatitis, endometriosis, neuropathic pain, hypersensitivity pneumonitis, eosinophilic pneumonias, delayed-type hypersensitivity, interstitial lung disease (ILD) (e.g., idiopathic pulmonary fibrosis, or ILD associated with rheumatoid arthritis, systemic lupus erythematosus, ankylosing spondylitis, systemic sclerosis, Sjogren's syndrome, polymyositis or dermatomyositis), eye diseases (e.g., retinal degeneration, choroidal neovascularization (CNV), etc.) and the like. Example immune disorders include rheumatoid arthritis, psoriatic arthritis, systemic lupus erythematosus, myastenia gravis, juvenile onset diabetes; glomerulonephritis, autoimmune throiditis, organ transplant rejection including allograft rejection and graft-versus-host disease. Example cancers include cancers such as breast cancer, ovarian cancer, multiple myeloma and the like that are characterized by infiltration of macrophages (e.g., tumor associated macrophages, TAMs) into tumors or diseased tissues.

As used herein, the term “contacting” refers to the bringing together of indicated moieties in an in vitro system or an in vivo system. For example, “contacting” the chemokine receptor with a salt of the invention includes the administration of a salt of the present invention to an individual or patient, such as a human, having a chemokine receptor, as well as, for example, introducing a salt of the invention into a sample containing a cellular or purified preparation containing the chemokine receptor.

As used herein, the term “individual” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent that elicits the biological or medicinal response that is being sought in a tissue, system, animal, individual or human by a researcher, veterinarian, medical doctor or other clinician, which includes one or more of the following:

(1) preventing the disease; for example, preventing a disease, condition or disorder in an individual who may be predisposed to the disease, condition or disorder but does not yet experience or display the pathology or symptomatology of the disease (non-limiting examples are preventing hypersensitivity lung diseases, drug-induced pulmonary fibrosis, chronic obstructive pulmonary disease (COPD), graft-versus-host disease and/or allograft rejection after transplantation, or preventing allergic reactions such as atopic dermatitis, delayed type hypersensitivity, or seasonal or perennial allergic rhinitis);

(2) inhibiting the disease and its progression; for example, inhibiting a disease, condition or disorder in an individual who is experiencing or displaying the pathology or symptomatology of the disease, condition or disorder (i.e., arresting further development of the pathology and/or symptomatology) such as inhibiting the inflammatory or autoimmune response in hypersensitivity lung diseases, drug-induced pulmonary fibrosis, chronic obstructive pulmonary disease (COPD), rheumatoid arthritis, lupus or psoriasis, or inhibitng progression of atherosclerotic plaques, Alzheimer's disease, macular degeneration or the progression of insulin resistance to a diabetic state, or inhibiting tumor growth or stabilizing viral load in the case of a viral infection; and

(3) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the autoimmune response in hypersensitivity lung diseases, drug-induced pulmonary fibrosis, chronic obstructive pulmonary disease (COPD), rheumatoid arthritis, lupus or psoriasis, or shrinking a tumor associated with cancer or lowering viral load in the case of a viral infection.

One or more additional pharmaceutical agents such as, for example, anti-viral agents, antibodies, anti-inflammatory agents, insulin secretagogues and sensitizers, serum lipid and lipid-carrier modulating agents, immunosuppressants, and/or chemotherapeutics can be used in combination with the compounds of the present invention for treatment of chemokine receptor-associated diseases, disorders or conditions. The agents can be combined with the present compounds in a single dosage form, or the agents can be administered simultaneously or sequentially as separate dosage forms.

Suitable antiviral agents contemplated for use in combination with the compounds of the present invention can comprise nucleoside and nucleotide reverse transcriptase inhibitors (NRTIs), non-nucleoside reverse transcriptase inhibitors (NNRTIs), protease inhibitors, entry inhibitors, fusion inhibitors, maturation inhibitors, and other antiviral drugs.

Example suitable NRTIs include zidovudine (AZT); didanosine (ddl); zalcitabine (ddC); stavudine (d4T); lamivudine (3TC); abacavir (1592U89); adefovir dipivoxil [bis(POM)-PMEA]; lobucavir (BMS-180194); BCH-10652; emitricitabine [(−)-FTC]; beta-L-FD4 (also called beta-L-D4C and named beta-L-2′, 3′-dicleoxy-5-fluoro-cytidene); DAPD, ((−)-beta-D-2,6,-diamino-purine dioxolane); and lodenosine (FddA).

Typical suitable NNRTIs include nevirapine (BI-RG-587); delaviradine (BHAP, U-90152); efavirenz (DMP-266); PNU-142721; AG-1549; MKC-442 (1-(ethoxy-methyl)-5-(1-methylethyl)-6-(phenylmethyl)-(2,4(1H,3H)-pyrimidi nedione); and (+)-calanolide A (NSC-675451) and B.

Typical suitable protease inhibitors include saquinavir (Ro 31-8959); ritonavir (ABT-538); indinavir (MK-639); nelfnavir (AG-1343); amprenavir (141W94); lasinavir (BMS-234475); DMP-450; BMS-2322623; ABT-378; and AG-1 549.

Other antiviral agents include hydroxyurea, ribavirin, IL-2, IL-12, pentafuside, enfuvirtide, C-34, the cyclotriazadisulfonamide CADA, PA-457, and Yissum Project No. 11607.

In some embodiments, anti-inflammatory or analgesic agents contemplated for use in combination with the compounds of the present invention can comprise, for example, an opiate agonist, a lipoxygenase inhibitor such as an inhibitor of 5-lipoxygenase, a cyclooxygenase inhibitor such as a cyclooxygenase-2 inhibitor, an interleukin inhibitor such as an interleukin-I inhibitor, a TNF inhibitor such as infliximab, etanercept, or adalimumab, an NNMA antagonist, an inhibitor of nitric oxide or an inhibitor of the synthesis of nitric oxide, a non-steroidal antiinflammatory agent, or a cytokine-suppressing antiinflammatory agent, for example, such as acetaminophen, asprin, codiene, fentanyl, ibuprofen, indomethacin, ketorolac, morphine, naproxen, phenacetin, piroxicam, a steroidal analgesic, sufentanyl, sunlindac, tenidap, and the like. Similarly, the instant compounds can be administered with a pain reliever; a potentiator such as caffeine, an H2-antagonist, simethicone, aluminum or magnesium hydroxide; a decongestant such as phenylephrine, phenylpropanolamine, pseudophedrine, oxymetazoline, ephinephrine, naphazoline, xylometazoline, propylhexedfine, or levo-desoxyephedrine; an antfitussive such as codeine, hydrocodone, caramiphen, carbetapentane, or dextramethorphan; a diuretic; and a sedating or non-sedating antihistamine.

In some embodiments, pharmaceutical agents contemplated for use in combination with the compounds of the present invention can comprise but are not limited to (a) VLA-4 antagonists such as those described in U.S. Pat. No. 5,510,332, W095/15973, W096/01644, W096/06108, W096/20216, W096/229661, W096/31206, W096/4078, W097/030941, W097/022897, WO 98/426567, W098/53814, W098/53817, W098/538185, W098/54207, and W098/58902; (b) steroids such as beclomethasone, methylpi-ednisolone, betamethasone, prednisone, dexamethasone, and hydrocortisone; (c) immunosuppressants such as cyclosporine, tacrolimus, rapamycin and other FK506 type immunosuppressants; (d) antihistamines (HI-histamine antagonists) such as bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine pyrilarnine, astemizole, terfenadine, loratadine, cetirizine, fexofenadine, desearboethoxyloratadine, and the like; (e) non-steroidal anti-asthmatics such as terbutaline, metaproterenol, fenoterol, isoethaiine, albuterol, bitolterol, pirbuterol, theophylline, cromolyn sodium, atropine, ipratropium bromide, leukotriene antagonists (e.g., zafirlukast, montelukast, pranlukast, iralukast, pobilukast, SKB-106,203), leukotriene biosynthesis inhibitors (e.g., zileuton, BAY-1005); (f) nonsteroidal antiinflammatory agents (NSAIDs) such as propionic acid derivatives (e.g., alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (e.g., indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac), fenamic acid derivatives (flufenamic acid, meclofenamic acid, mefenamic acid, niflumic acid and tolfenamic acid), biphenylearboxylic acid derivatives (diflunisal and flufenisal), oxicams (isoxicam, piroxicam, sudoxicam and tenoxican), salicylates (acetyl salicylic acid, sulfasalazine) and the pyrazolones (apazone, bezpiperylon, feprazone, mofebutazone, oxyphenbutazone, phenylbutazone); (g) cyclooxygenase-2 (COX-2) inhibitors; (h) inhibitors of phosphodiesterase type IV (PDE-IV); (i) other antagonists of the chemokine receptors, especially CXCR-4, CCRI, CCR2, CCR3 and CCR5; (j) cholesterol lowering agents such as HMG-CoA reductase inhibitors (lovastatin, sirrivastatin and pravastatin, fluvastatin, atorvastatin, and other statins), sequestrants (cholestyramine and colestipol), nicotinic acid, fenofibric acid derivatives (gemfibrozil, clofibrat, fenofibrate and benzafibrate), and probucol; (k) anti-inflammatory biologic agents such as anti-TNF therapies, anti-IL-1 receptor, CTLA-4Ig, anti-CD20, and anti-VLA4 antibodies; (l) anti-diabetic agents such as insulin, sulfonylureas, biguanides (metformin), U.-glucosidase inhibitors (acarbose) and orlitazones (troglitazone and pioglitazone); (m) preparations of interferon beta (interferon beta- lo., interferon beta-1 P); (n) other compounds such as aminosalicylic acids, antimetabolites such as azathioprine and 6-mercaptopurine, and cytotoxic cancer chemotherapeutic agents. The weight ratio of the compound of the compound of the present invention to the second active ingredient may be varied and will depend upon the effective dose of each ingredient.

For example, a CCR2 antagonist can be used in combination with an anti-inflammatory pharmaceutical agent in the treatment of inflammation, metabolic disease, autoimmune disease, cancer or viral infection to improve the treatment response as compared to the response to the anti-inflammatory agent alone, without exacerbation of its toxic effects. Additive or synergistic effects are desirable outcomes of combining a CCR2 antagonist of the present invention with an additional agent. Furthermore, resistance of cancer cells to. agents such as dexamethasone can be reversible upon treatment with a CCR2 antagonist of the present invention.

Pharmaceutical Formulations and Dosage Forms

When employed as pharmaceuticals, the salts of the invention can be administered in the form of pharmaceutical compositions. These compositions can be prepared in a manner well known in the pharmaceutical art, and can be administered by a variety of routes depending upon whether local or systemic treatment is desired and upon the area to be treated. Administration can be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or can be, for example, by a continuous perfusion pump. Pharmaceutical compositions and formulations for topical administration can include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

This invention also includes pharmaceutical compositions which contain, as the active ingredient, one or more of the salts above in combination with one or more pharmaceutically acceptable carriers. In making the compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

The compositions can be formulated in a unit dosage form, each dosage containing from about 5 to about 1000 mg (1 g), more usually about 100 to about 500 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, 0.1 to about 1000 mg of the active ingredient of the present invention.

The tablets or pills of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions in can be nebulized by use of inert gases. Nebulized solutions may be breathed directly from the nebulizing device or the nebulizing device can be attached to a face masks tent, or intermittent positive pressure breathing machine. Solution, suspension, or powder compositions can be administered orally or nasally from devices which deliver the formulation in an appropriate manner.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of the compounds of the present invention can vary according to, for example, the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. For example, the salts of the invention can be provided in an aqueous physiological buffer solution containing about 0.1 to about 10% w/v of the compound for parenteral adminstration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The salts of the invention can also be formulated in combination with one or more additional active ingredients which can include any pharmaceutical agent such as antibodies, immune suppressants, anti-inflammatory agents, chemotherapeutics, lipid lowering agents, HDL elevating agents, insulin secretagogues or sensitizers, and drugs used for the treatment of rheumatoid arthritis and the like.

Rheumatoid Arthritis (RA) Treatment Regimen

Rheumatoid arthritis (RA) patients, treated aggressively with disease modifying agents (methotrexate, antimalarials, gold, penicillamine, sulfasalazine, dapsone, leflunamide, or biologicals), can achieve varying degrees of disease control, including complete remissions. These clinical responses are associated with improvement in standardized scores of disease activity, specifically the ACR criteria which includes: pain, function, number of tender joints, number of swollen joints, patient global assessment, physician global assessment, laboratory measures of inflammation (CRP and ESR), and radiologic assessment of joint structural damage. Current disease-modifying drugs (DMARDs) require continued administration to maintain optimal benefit. Chronic dosing of these agents is associated with significant toxicity and host defense compromise. Additionally, patients often become refractory to a particular therapy and require an alternative regimen. For these reasons, a novel, effective therapy which allows withdrawal of standard DMARDs would be a clinically important advance.

Patients with significant response to anti-TNF therapies (infliximab, etanercept, adalimumab), anti- IL-1 therapy (kinaret) or other disease modifying anti-rheumatic drugs (DMARDs) including but not limited to methotrexate, cyclosporine, gold salts, antimalarials, penicillamine or leflunamide, who have achieved clinical remission of disease can be treated with a substance that inhibits expression and/or activity of CCR2 including, for example, nucleic acids (e.g., antisense or siRNA molecules), proteins (e.g., anti-CCR2 antibodies), small molecule inhibitors (e.g., the compounds disclosed herein and other chemokine receptor inhibitors known in the art).

In some embodiments, the substance that inhibits expression and/or activity of CCR2 is a small molecule CCR2 inhibitor (or antagonist). The CCR2 antagonist can be dosed orally q.d. or b.i.d at a dose not to exceed about 500 mgs a day. The patients can be withdrawn from or have a decrease in the dosage of their current therapy and would be maintained on treatment with the CCR2 antagonist. Treating patients with a combination of CCR2 antagonist and their current therapy can be carried out for, for example, about one to about two days, before discontinuing or dose reducing the DMARD and continuing on CCR2 antagonist.

Advantages of substituting traditional DMARDS with CCR2 antagonists are numerous. Traditional DMARDs have serious cumulative dose-limiting side effects, the most common being damage to the liver, as well as immunosuppressive actions. CCR2 antagonism is expected to have an improved long-term safety profile and will not have similar immunosuppressive liabilities associated with traditional DMARDs. Additionally, the half-life of the biologicals is typically days or weeks, which is an issue when dealing with adverse reactions. The half-life of an orally bioavailable CCR2 antagonist is expected to be on the order of hours so the risk of continued exposure to the drug after an adverse event is very minimal as compared to biological agents. Also, the current biologic agents (infliximab, etanercept, adalimumab, kinaret) are typically given either i.v. or s.c., requiring doctor's administration or patient self-injection. This leads to the possibility of infusion reaction or injection site reactions. These are avoidable using an orally administered CCR2 antagonist.

Diabetes and Insulin Resistance Treatment Regimen

Type 2 diabetes is one of the leading causes of morbidity and mortality in western societies. In the vast majority of patients, the disease is characterized by pancreatic beta-cell dysfunction accompanied by insulin resistance in the liver and in peripheral tissues. Based on the primary mechanisms that are associated with disease, two general classes of oral therapies are available to treat type 2 diabetes: insulin secretagogues (sulfonylureas such as glyburide) and insulin sensitizers (metformin and thiazolidinediones such as rosiglitazone). Combination therapy that addresses both mechanisms has been shown to manage the metabolic defects of this disease and in many instances can be shown to ameliorate the need for exogenous insulin administration. However, with time, insulin resistance often progresses, leading to the need for further insulin supplementation. In addition, a prediabetic state, referred to as the metabolic syndrome, has been demonstrated to be characterized by impaired glucose tolerance, particularly in association with obesity. The majority of patients who develop type 2 diabetes begin by developing insulin resistance, with the hyperglycemia occurring when these patients can no longer sustain the degree of hyperinsulinemia necessary to prevent loss of glucose homeostasis. The onset of the insulin resistance component is highly predictive of disease onset and is associated with an increase in the risk of developing type 2 diabetes, hypertension and coronary heart disease.

One of the strongest correlates of impaired glucose tolerance and of the progression from an insulin resistant state to type 2 diabetes is the presence of central obesity. Most patients with type 2 diabetes are obese and obesity itself is associated with insulin resistance. It is clear that central adiposity is a major risk factor for the development of insulin resistance leading to type 2 diabetes, suggesting that signals from visceral fat contribute to the development of insulin resistant and progression to disease. In addition to the secreted protein factors, obesity induces a cellular inflammatory response in which bone-marrow derived macrophages accumulate in adipose depots, becoming adipose tissue macrophages. Adipose tissue macrophages accumulate in adipose tissue in proportion to measures of adiposity. Tissue infiltrating macrophages are a source of many of the inflammatory cytokines that have been demonstrated to induce insulin resistance in adipocytes.

Adipose tissue produces MCP-1 in proportion to adiposity, suggesting that its activity by signaling through CCR2 also might play an important role in the accumulation of macrophages in adipose tissue. It is unknown whether the MCP-I/CCR2 interaction is directly responsible for monocyte recruitment to adipose tissue, whether reduced recruitment of macrophages to adipose tissue in humans will directly lead to the reduced production of proinflammatory molecules and whether the proinflammatory molecule production is directly linked to insulin resistance.

Patients who demonstrate insulin resistance, either prediabetic (normoglycemic) or diabetic (hyperglycemic), could be treated with a substance that inhibits the expression and/or activity of CCR2 including, for example, nucleic acids (e.g., antisense or siRNA molecules), proteins (e.g., anti-CCR2 antibodies), small molecule inhibitors (e.g., the compounds disclosed herein and other chemokine receptor inhibitors known in the art). In some embodiments, the substance that inhibits expression and/or activity of CCR2 is a small molecule CCR2 inhibitor (or antagonist). The CCR2 antagonist can be dosed orally q.d. or b.i.d at a dose not to exceed about 500 mgs a day. The patients can be withdrawn from or have a decrease in the dosage of their current therapy and would be maintained on treatment with the CCR2 antagonist. Alternately CCR2 antagonist treatment may be used to supplement their current therapy to enhance its effectiveness or to prevent progression to further insulin dependence.

Advantages of substituting or supplementing traditional agents with CCR2 antagonists are numerous. Such agents may be useful, for example, to preclude progression from a prediabetic, insulin resistant state to a diabetic state. Such agents may reduce or replace the need for the use of insulin sensitizers, with their attendant toxicities. Such agents may also reduce the need for, or prolong the period until, exogenous insulin supplementation is required.

Atherosclerosis Treatment Regimen

Atherosclerosis is a condition characterized by the deposition of fatty substances in arterial walls. Plaque encompasses such deposits of fatty substances, cholesterol, cellular waste products, calcium and other substances that build up in the inner lining of an artery. Plaques can grow large enough to significantly reduce the blood's flow through an artery. However, more significant damage occurs when the plaque becomes unstable and ruptures. Plaques that rupture cause blood clots to form that can block blood flow or break off and travel to other parts of the body. If the clot blocks a blood vessel that feeds the heart, it causes a heart attack. If it blocks a blood vessel that feeds the brain, it causes a stroke. Atherosclerosis is a slow, complex disease that typically starts in childhood and often progresses as people grow older.

A high level of cholesterol in the blood is a major risk factor for coronary heart disease. Based on cholesterol as a primary composition of plaque, the advance of plaque formation has been managed by the reduction of circulating cholesterol or by elevation of cholesterol-carrying high density lipoproteins (HDL). Circulating cholesterol can be reduced, for example, by inhibiting its synthesis in the liver using or by reducing update from food. Such medicaments that act through these mechanism may include medicines that are used to lower high cholesterol levels: bile acid absorbers, lipoprotein synthesis inhibitors, cholesterol synthesis inhibitors and fibric acid derivatives. Circulating HDL can additionally be elevated by administration of, for example, probuchol or high doses of niacin. Therapy that addresses multiple mechanisms has been shown to slow disease progression and progression to plaque rupture.

Atherosclerosis is typically accompanied by a cellular inflammatory response in which bone-marrow derived macrophages accumulate in fatty streaks along the vessel wall, becoming foam cells. Foam cells are a source of many of the inflammatory cytokines that have been demonstrated to induce plaque progression and of the enzymes that can promote plaque destabilization. Atherosclerotic tissue also produces MCP-1, suggesting that its activity by signaling through CCR2 also might play an important role in the accumulation of macrophages as foam cells in plaques. CCR2−/− mice have been demonstrated to have significantly reduced macrophages in fatty streaks generated as a result of high fat diet or genetic alteration in lipid metabolism.

Patients who demonstrate high circulating cholesterol, low HDL, or elevated circulating CRP or present with vessel wall plaque by imaging, or any other evidence of the presence of atherosclerosis could be treated with a substance that inhibits the expression and/or activity of CCR2 including, for example, nucleic acids (e.g., antisense or siRNA molecules), proteins (e.g., anti-CCR2 antibodies), small molecule inhibitors (e.g., the compounds disclosed herein and other chemokine receptor inhibitors known in the art). In some embodiments, the substance that inhibits expression and/or activity of CCR2 is a small molecule CCR2 inhibitor (or antagonist) such as a compound of the invention. The CCR2 antagonist can be dosed orally q.d. or b.i.d at a dose not to exceed about 500 mgs a day. The patients can be withdrawn from or have a decrease in the dosage of their current therapy and would be maintained on treatment with the CCR2 antagonist. Alternately CCR2 antagonist treatment may be used to supplement their current therapy to enhance its effectiveness in, for example, preventing plaque progression, stabilizing plaque that has already formed or inducing plaque regression.

Advantages of substituting or supplementing traditional agents with CCR2 antagonists are numerous. Such agents may be useful, for example, to preclude progression of the plaque to a stage of instability with its associated risk of plaque rupture. Such agents may reduce or replace the need for the use of cholesterol modifying drugs or HDL elevating drugs, with their attendant toxicities including, but not limited to, flushing, liver damage and muscle damage such as myopathy. Such agents may also reduce the need for, or prolong the period until, surgery is required to open the vessel wall or until use of anticoagulants is required to limit damage due to potential plaque rupture.

Labeled Compounds and Assay Methods

Another aspect of the present invention relates to labeled salts of the invention (radio-labeled, fluorescent-labeled, etc.) that would be useful not only in radio-imaging but also in assays, both in vitro and in vivo, for localizing and quantitating the enzyme in tissue samples, including human, and for identifying ligands by inhibition binding of a labeled compound. Accordingly, the present invention includes enzyme assays that contain such labeled salts.

An “isotopically” or “radio-labeled” salt is a salt of the invention where one or more atoms are replaced or substituted by an atom having an atomic mass or mass number different from the atomic mass or mass number typically found in nature (i.e., naturally occurring). Suitable radionuclides that may be incorporated in compounds of the present invention include but are not limited to ²H (also written as D for deuterium), ³H (also written as T for tritium), ¹¹C, ¹¹C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is incorporated in the instant radio-labeled compounds will depend on the specific application of that radio-labeled compound. For example, for in vitro chemokine receptor labeling and competition assays, compounds that incorporate ³H, ¹⁴C, ⁸²Br, ¹²⁵I, ¹³¹I, ³⁵S or will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful.

It is understood that a “radio-labeled” or “labeled compound” is a compound that has incorporated at least one radionuclide. In some embodiments the radionuclide is selected from the group consisting of ³H, ¹⁴C, ¹²⁵I , ³⁵S and ⁸²Br.

Synthetic methods for incorporating radio-isotopes into organic compounds are applicable to compounds of the invention and are well known in the art.

A radio-labeled salt of the invention can be used in a screening assay to identify/evaluate compounds. In general terms, a newly synthesized or identified compound (i.e., test compound) can be evaluated for its ability to reduce binding of the radio-labeled salt of the invention to the chemokine receptor. Accordingly, the ability of a test compound to compete with the radio-labeled compound for binding to the chemokine receptor directly correlates to its binding affinity.

Kits

The present invention also includes pharmaceutical kits useful, for example, in the treatment or prevention of chemokine-associated diseases which include one or more containers containing a pharmaceutical composition comprising a therapeutically effective amount of a salt of the invention. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

It is appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### EXAMPLES

### Example 1

### Preparation of Bis(Methanesulfonic Acid) Salt

Methanesulfonic acid (1.729 g, 17.99 mmol) in methyl isobutyl ketone (18.00 mL) was added slowly to a solution of N-({(R)-1-[trans-4-hydroxy-4-(6-methoxy-pyridin-3-yl)-cyclohexyl]-pyrrolidin-3-ylcarbamoyl}-methyl)-3-trifluoromethyl-benzamide (4.50 g, 8.64 mmol) in isopropyl alcohol (20.25 mL) and water (2.25 mL) with stirring. After addition, methyl isobutyl ketone (21.37 mL) was added slowly to the above mixture until it became slightly cloudy. A slurry was formed after 30 min at which time additional methyl isobutyl ketone (22.00 mL) was added and the reaction mixture was stirred at room temperature overnight. The white precipitate was collected by vacuum filtration and the cake was washed with methyl isobutyl ketone/isopropanol (v/v=3:1, 22 mL). The cake was dried under high vacuum to provide 5.23 g (85%) of N-[2-({(3R)-1-[4-hydroxy-4-(6-methoxypyridin-3-yl)cyclohexyl]pyrrolidin-3-yl}amino)-2-oxoethyl]-3-(trifluoromethyl)benzamide dimethanesulfonate as a crystalline solid.

### Example 2

### Properties of the Bis(Methanesulfonic Acid) Salt

A crystalline sample of N-[2-({(3R)-1-[4-hydroxy-4-(6-methoxypyridin-3-yl)cyclohexyl]pyrrolidin-3-yl}amino)-2-oxoethyl]-3-(trifluoromethyl)benzamide dimethanesulfonate, prepared in a manner substantially according to Example 1, was shown to have the properties provided in Table 4. DSC and XRPD data are provided in FIGS. 1 and 2.

TABLE 4

| | |
|---|---|
| Elemental Analysis | Calc'd: C, 47.18; H, 5.52; N, 7.86; S, 9.00 |
| | Found: C, 47.00; H, 5.49; N, 7.48; S, 9.56 |
| Water Content (Karl Fisher) | 0.41% |
| DSC (° C.) | 163 (onset); 166 (peak) |

### Example 3

### Recrystallization of Bis(Methanesulfonic Acid) Salt

N-[2-({(3R)-1-[trans-4-hydroxy-4-(6-methoxypyridin-3-yl)cyclohexyl]pyrrolidin-3-yl}amino)-2-oxoethyl]-3-(trifluoromethyl)benzamide dimethanesulfonate (15.0 g, 21.04 mmol) was dissolved in isopropyl alcohol (45.00 mL) and water (5.00 mL) with stirring at room temperature for 2-3 h to give a clear solution. Methyl isobutyl ketone (100 mL) was added slowly to the above mixture until it became slightly cloudy. A slurry was formed after stirring for 1 h at which time additional methyl isobutyl ketone (80.0 mL) was added and the reaction mixture was stirred at room temperature for 4 h. The white precipitate was collected by vacuum filtration and the cake was washed with methyl isobutyl ketone/isopropanol (v/v=3:1, 25 mL). The cake was dried under high vacuum to provide 13.0 g (87%) of N-[2-({(3R)-1-[4-hydroxy-4-(6-methoxypyridin-3-yl)cyclohexyl]pyrrolidin-3-yl}amino)-2-oxoethyl]-3-(trifluoromethyl)benzamide dimethanesulfonate as a crystalline solid.

### Example 4

### Preparation of Bis(Ethanesulfonic Acid) Salt

A solution of ethanesulfonic acid (53.43 mg, 0.4803 mmol) in isopropyl alcohol (2.00 mL) was added to a solution of N-({(R)-1-[trans-4-hydroxy-4-(6-methoxy-pyridin-3-yl)-cyclohexyl]-pyrrolidin-3-ylcarbamoyl}-methyl)-3-trifluoromethyl-benzamide (100 mg, 0.192 mmol) in isopropanol (2.0 mL) at room temperature. After being stirred for 20 min, a white slurry was formed. Stirring was continued for 4 h at room temperature and the slurry was filtered under vacuum and washed with isopropanol. The resulting cake was dried under high vacuum overnight to provide 43.0 mg (30%) of N-[2-({(3R)-1-[trans-4-hydroxy-4-(6-methoxypyridin-3-yl)cyclohexyl]pyrrolidin-3-yl}amino)-2-oxoethyl]-3-(trifluoromethyl)-benzamide diethanesulfonate as a crystalline solid. DSC and XRPD spectra are provided in FIGS. 3 and 4, respectively.

### Example 5

### Properties of the Bis(Ethanesulfonic Acid) Salt

A crystalline sample of N-[2-({(3R)-1-[4-hydroxy-4-(6-methoxypyridin-3-yl)cyclohexyl]pyrrolidin-3-yl}amino)-2-oxoethyl]-3-(trifluoromethyl)benzamide diethanesulfonate, prepared in a manner substantially according to Example 4, was shown to have the properties provided in Table 5. DSC and XRPD data are provided in FIGS. 3 and 4.

TABLE 5

| | |
|---|---|
| Elemental Analysis | Calc'd: C, 48.64; H, 5.85; |
| | N, 7.56; F, 7.69; S, 8.66 |
| | Found: C, 48.45; H, 5.72; |
| | N, 7.47; F, 7.60; S, 8.95 |
| Water Content (Karl Fisher) | 0.27% |
| DSC (° C.) | 170 (onset); 173 (peak) |

### Example 6

### Preparation of Camphoric Acid Salt

(1R,3S)-1,2,2-Trimethylcyclopentane-1,3-dicarboxylic acid (0.846 g, 4.23 mmol) in ethyl acetate (20.0 mL) was slowly added to a solution of N-[2-({(3R)-1-[trans-4-hydroxy-4-(6-methoxypyridin-3-yl)cyclohexyl]pyrrolidin-3-yl}amino)-2-oxoethyl]-3-(trifluoromethyl)-benzamide (2.00 g, 3.84 mmol) in ethyl acetate (20.0 mL). After being stirred at room temperature for 4 h, the white precipitate was collected by vacuum filtration and the cake was washed with EtOAc (10 mL). The cake was dried under vacuum to constant weight to provide 2.46 g (89%) of N-[2-({(3R)-1-[trans-4-hydroxy-4-(6-methoxypyridin-3-yl)cyclohexyl]pyrrolidin-3-yl}amino)-2-oxoethyl]-3-(trifluoromethyl)benzamide (1R,3S)-1,2,2-trimethylcyclopentane-1,3-dicarboxylate as a white crystalline solid. DSC and XRPD spectra are provided in FIGS. 5 and 6, respectively.

### Example 7

### Properties of the Camphoric Acid Salt

A crystalline sample of N-[2-({(3R)-1-[4-hydroxy-4-(6-methoxypyridin-3-yl)cyclohexyl]pyrrolidin-3-yl}amino)-2-oxoethyl]-3-(trifluoromethyl)benzamide camphorate, prepared in a manner substantially according to Example 6, was shown to have the properties provided in Table 6. DSC and XRPD data are provided in FIGS. 5 and 6.

TABLE 6

| | |
|---|---|
| Elemental Analysis | Calc'd: C, 59.99; H, 6.57; N, 7.77; F, 7.91 |
| | Found: C, 59.69; H, 6.43; N, 7.56; F, 8.02 |
| Water Content (Karl Fisher) | 0.18% |
| DSC (° C.) | 173 (onset); 176 (peak) |

Various modifications of the invention, in addition to those described herein, will be apparent to those skilled in the art from the foregoing description. Such modifications are also intended to fall within the scope of the appended claims. Each reference, including patents, patent applications, and publications, cited in the present application is incorporated herein by reference in its entirety.

What is claimed is:

1. A pharmaceutically acceptable salt of a compound of Formula I: 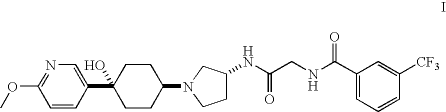
wherein said salt is a bis(methanesulfonic acid) salt, bis(ethanesulfonic acid) salt, or camphoric acid salt.

2. The salt of claim 1 wherein said salt is a bis(methanesulfonic acid) salt.

3. The salt of claim 1 wherein said salt is a bis(ethanesulfonic acid) salt.

4. The salt of claim 1 wherein said salt is a camphoric acid salt.

5. The salt of claim 1 wherein said salt is crystalline.

6. The salt of claim 1 wherein said salt is anhydrous.

7. The salt of claim 1, wherein said salt is a bis(methanesulfonic acid) salt having a DSC thermogram substantially as shown in FIG. 1.

8. The salt of claim 1, wherein said salt is a bis(methanesulfonic acid) salt having a DSC endotherm peak at about 166° C.

9. The salt of claim 1, wherein said salt is a bis(methanesulfonic acid) salt having an X-ray powder diffraction pattern substantially as shown in FIG. 2.

10. The salt of claim 1, wherein said salt is a bis(methanesulfonic acid) salt having an X-ray powder diffraction pattern comprising peaks, in terms of 2θ, at about 8.7° and about 21.8°.

11. The salt of claim 1, wherein said salt is a bis(methanesulfonic acid) salt having an X-ray powder diffraction pattern comprising peaks, in terms of 2θ, at about 8.7°, about 21.8°, about 20.1°, and about 20.9°.

12. The salt of claim 1, wherein said salt is a bis(methanesulfonic acid) salt having an X-ray powder diffraction pattern comprising peaks, in terms of 2θ, at about 8.7°, about 21.8°, about 20.1°, about 20.9°, about 22.5°, and about 17.2°.

13. The salt of claim 1, wherein said salt is a bis(ethanesulfonic acid) salt having a DSC thermogram substantially as shown in FIG. 3.

14. The salt of claim 1, wherein said salt is a bis(ethanesulfonic acid) salt having a DSC endotherm peak at about 173° C.

15. The salt of claim 1, wherein said salt is a bis(methanesulfonic acid) salt having an X-ray powder diffraction pattern substantially as shown in FIG. 4.

16. The salt of claim 1, wherein said salt is a bis(methanesulfonic acid) salt having an X-ray powder diffraction pattern comprising at least one peak, in terms of 2η, at about 9.2°.

17. The salt of claim 1, wherein said salt is a bis(methanesulfonic acid) salt having an X-ray powder diffraction pattern comprising peaks, in terms of 2θ, at about 9.20, about 12.1°, and about 18.3°.

18. The salt of claim 1, wherein said salt is a bis(methanesulfonic acid) salt having an X-ray powder diffraction pattern comprising peaks, in terms of 2θ, at about 9.20, about 12.1°, about 13.8°, about 18.3°, about 19.3°, and about 19.8°.

19. The salt of claim 1, wherein said salt is a camphoric acid salt having a DSC thermogram substantially as shown in FIG. 5.

20. The salt of claim 1, wherein said salt is a bis(ethanesulfonic acid) salt having a DSC endotherm peak at about 176° C.

21. The salt of claim 1, wherein said salt is a camphoric acid salt having an X-ray powder diffraction pattern substantially as shown in FIG. 6.

22. The salt of claim 1, wherein said salt is a camphoric acid salt having an X-ray powder diffraction pattern comprising peaks, in terms of 2θ, at about 17.0° and about 19.1°.

23. The salt of claim 1, wherein said salt is a camphoric acid salt having an X-ray powder diffraction pattern comprising peaks, in terms of 2θ, at about 17.0°, about 19.1°, about 17.8°, and about 14.1°.

24. The salt of claim 1, wherein said salt is a camphoric acid salt having an X-ray powder diffraction pattern comprising peaks, in terms of 2θ, at about 17.0°, about 19.1°, about 17.8°, about 14.1°, about 16.3°, and about 18.4°.

25. The salt of claim 1, wherein said salt is a camphoric acid salt having an X-ray powder diffraction pattern comprising peaks, in terms of 2θ, at about 17.0°, about 19.1°, about 17.8°, about 14.1°, about 16.3°, about 18.4°, about 10.1° and about 11.7°.

26. A method of preparing the salt of claim 1, wherein said salt is a bis(methanesulfonic acid) salt, comprising:
combining said compound of Formula I with methane sulfonic acid in a crystallizing solvent comprising water, alcohol, and ketone; and
precipitating said salt from said crystallizing solvent.

27. The method of claim 26 wherein said alcohol comprises isopropanol.

28. The method of claim 26 wherein said ketone comprises methyl isobutyl ketone.

29. The method of claim 26 wherein said precipitating is induced by adding ketone to said crystallizing solvent.

30. The method of claim 26 wherein the volume ratio of water to alcohol in said crystallizing solvent is about 1:2 to about 1:20.

31. The method of claim 26 wherein the volume ratio of water to alcohol in said crystallizing solvent is about 1:5 to about 1:12.

32. The method of claim 26 wherein the volume ratio of water to alcohol in said crystallizing solvent is about 1:9.

33. A salt prepared by the method of claim 26.

34. A method of preparing the salt of claim 1, wherein said salt is a bis(ethanesulfonic acid) salt, comprising:
combining said compound of Formula I with ethane sulfonic acid in a crystallizing solvent comprising an alcohol; and
precipitating said salt from said crystallizing solvent.

35. The method of claim 34 wherein said alcohol comprises isopropanol.

36. A salt prepared by the method of claim 34.

37. A method of preparing the salt of claim 1, wherein said salt is a camphoric acid salt, comprising:
combining said compound of Formula I with camphoric acid in a crystallizing solvent comprising ethyl acetate; and
precipitating said salt from said crystallizing solvent.

38. A salt prepared by the method of claim 37.

39. A composition comprising the salt of claim 1 and a pharmaceutically acceptable carrier.

40. A method of modulating activity of a chemokine receptor comprising contacting said chemokine receptor with a salt of claim 1.

41. The method of claim 40 wherein said chemokine receptor is CCR2.

42. The method of claim 40 wherein said modulating corresponds to inhibiting.

43. A method of treating a disease associated with expression or activity of a chemokine receptor in a patient comprising administering to said patient a therapeutically effective amount of a salt of claim 1.

44. The method of claim 43 wherein said chemokine receptor is CCR2.

45. The method of claim 43 wherein said disease is an inflammatory disease.

46. The method of claim 43 wherein said disease is an immune disorder.

47. The method of claim 43 wherein said disease is rheumatoid arthritis, atherosclerosis, lupus, multiple sclerosis, neuropathic pain, transplant rejection, diabetes, or obesity.

48. The method of claim 43 wherein said disease is cancer.

49. The method of claim 48 wherein said cancer is characterized by tumor associated macrophages.

50. The method of claim 48 wherein said cancer is breast cancer, ovarian cancer or multiple myeloma.

51. The method of claim 43 further comprising administering an anti-inflammatory agent.

52. The method of claim 51 wherein said anti-inflammatory agent is an antibody.
